(19) United States
(12) Patent Application Publication
(10) Pub. No.: US 2010/0121424 A1
(43) Pub. Date: May 13, 2010

(54) Stent compression tool

(76) Inventors: Petr Kubena, Hradec Kralove (CZ); Dobromil Sejbl, Hradec Kralove (CZ)

Correspondence Address: LITMAN LAW OFFICES, LTD., POST OFFICE BOX 41200, SOUTH STATION, ARLINGTON, VA, 22204, US

(21) Appl. No.: 12/292,142

(22) Filed: Nov. 12, 2008

(51) Int. Cl. A61F 2/06

(52) U.S. Cl. 623/1.11

(57) ABSTRACT

The stent compression tool is used to install a diametrically compressible stent in a delivery catheter. The tool has a tubular configuration with a larger funnel mouth at one end and a narrower opening at the opposite end. The basic steps in the process comprise extending the stent carrying member from its catheter sheath, passing the carrying member through the tool from the narrower end thereof, placing the stent over the carrying member and compressing the stent by pushing it through the funnel mouth of the tool and the narrower lumen of the tool, inserting the stent grip member of the delivery catheter into the stent and further into the tool, pushing the end of the sheath into the end of the tool, using the carrying member and its stent grip to draw the compressed stent from the tool into the sheath, and removing the tool from the carrying member.

### BACKGROUND OF THE INVENTION

1. Field of the Invention

The present invention relates generally to medical devices and equipment, and more particularly to a stent compression tool for diametrically compressing a stent and placing the stent within a stent delivery catheter. A method of stent compression using the tool is also disclosed.

2. Description of the Related Art

The surgical installation of stents, i.e., tubular devices used to repair or maintain an open passage through a bodily vessel, has been known and practiced for a considerable time. Stents are almost universally delivered and installed in the desired location by means of a stent delivery catheter, i.e., a hollow flexible tube or sheath carrying the stent therein. The smallest practicable diameter is most desirable for the delivery catheter, in order to avoid undue trauma to the patient. Yet, the stent itself must be of sufficient diameter to completely fill the diameter of the vessel in which it is installed.

Obviously, some means of placing the stent within the sheath is required. In the case of self-expanding stents, the stent must be compressed diametrically for insertion within the relatively smaller diameter of the delivery catheter sheath. This can be a tedious operation, depending upon the configuration of the stent and delivery catheter. As a result, many stents are pre-installed within the delivery catheter at the time of manufacture of the stent and catheter, thus precluding need for the medical practitioner (e.g., surgeon or nurse) to take the time to install the stent at the time of the stent implant operation.

Stents having various characteristics have been developed over the years, e.g., self-expanding and balloon expandable, biodegradable, woven, braided, or mesh construction using natural or synthetic materials, etc. Some of the more desirable materials for stent manufacture are biodegradable, allowing the stent to be absorbed or to dissolve within the body over a period of time to preclude requirement for subsequent additional surgery for stent removal in the case of a temporary implant.

However, a problem with many stents, and particularly those formed of biodegradable material, is that such material tends to take a “set,” i.e., to retain the physical shape and configuration to which it has been subjected, if held in that shape and configuration for long. This characteristic does not allow such stents to be installed within the delivery catheter long before the operation. In the case of stents formed of materials that tend to take a set, the stent is generally installed within the delivery catheter immediately before the operation. This can be a tedious procedure, as noted above, and in any case it takes further time to carry out the stent-to-catheter installation process, with time often being critical in the performance of medical procedures.

The present inventors are aware of a number of different devices that have been developed in the past for the installation of stents within bodily vessels. An example of such is Czech Republic Patent Document No. 14,595, filed on May 24, 2004. The first named inventor in the '595 Czech Republic reference is also the first named inventor of the present stent compression tool. The '595 Czech reference describes a series of flexible concentric tubes, with the self-expanding stent being placed within the outermost or largest diameter tube. The assembly is used to implant the stent at the desired location by sliding the outermost tube axially from its position over the stent and intermediate tube when the stent has been properly located, and withdrawing the remainder of the delivery assembly. No stent compression tool for loading the stent into a delivery catheter is described in this document.

None of the above-described inventions and patents, taken either singly or in combination, is seen to describe the stent compression tool as claimed. Thus, a stent compression tool solving the aforementioned problems is desired.

### SUMMARY OF THE INVENTION

The stent compression tool includes a generally tubular device having a funnel-shaped mouth at one end and a relatively narrower opening at the opposite end. The narrower end portion or sleeve includes a series of longitudinal slots to allow the sleeve to expand over the atraumatic tip of the delivery catheter assembly during the stent installation process. A sleeve retaining collar slides axially along the sleeve to preclude spreading of the sleeve segments or to allow them to spread, depending upon the collar position along the sleeve. A collar stop extends axially from the collar to prevent inadvertent separation of the collar from the tube.

The stent compression tool is used with a conventional stent delivery catheter to install the stent within the end of the catheter. The tool is initially installed over the extended atraumatic tip, i.e., a relatively large diameter and blunt tip configured to minimize puncture or other trauma to the bodily passage into which the delivery catheter is inserted, and stent carrying member of the catheter assembly, and the stent is inserted through the larger diameter funnel mouth or opening of the tool. The stent is pushed through the tool, whereupon it is compressed diametrically as it passes through the narrower sleeve portion of the tool. When a portion of the stent has extended from the narrow end or opening of the tool, the stent carrying member with its stent grip is inserted farther through the tool for the grip to engage the inner diameter of the stent and pull it back into the narrow portion of the tool.

At this point, the outer sheath of the delivery catheter is inserted into the narrow end of the tool until it engages a slightly smaller diameter sheath stop. The inner diameter of the sleeve portion of the tool beyond the sheath stop and the inner diameter of the sheath are essentially equal. The sheath carrying member with its stent grip is then withdrawn back into the catheter sheath, pulling the compressed stent into the sheath as well. The carrying member with its atraumatic tip is then pulled back through the lumen or passage of the tool to remove the tool from the catheter assembly, and the carrying member is drawn back through the catheter sheath to position the atraumatic tip at the distal or working end of the catheter sheath to complete the process.

These and other features of the present invention will become readily apparent upon further review of the following specification and drawings.

### BRIEF DESCRIPTION OF THE DRAWINGS

FIG. 1 is a perspective view of a stent compression tool according to the present invention, shown with the sleeve retaining collar secured over the split sleeve of-the device and the collar limit stop retracted.

FIG. 2 is a perspective view of the stent compression tool of FIG. 1, shown with the sleeve retaining collar retracted to allow the split sleeve to open and with the limit stop extended.

FIG. 3 is an environmental side elevation view of the stent compression tool in the configuration of FIG. 2, showing the tool ready for installation over the atraumatic tip and carrying member of a stent delivery catheter.

FIG. 4 is a detailed environmental side elevation view in section of the stent compression tool of FIGS. 1-2 being passed over the atraumatic tip of the carrying member.

FIG. 5 is a detailed environmental side elevation view in section, showing the stent compression tool of FIGS. 1-2 being passed beyond the atraumatic tip of the carrying member and the sleeve retaining collar moved to secure the split sleeve in its closed position.

FIG. 6 is a detailed environmental side elevation view in section showing the stent partially inserted through the stent compression tool of FIGS. 1-2, the stent being partially compressed and disposed over the atraumatic tip and end of the carrying member within the tool.

FIG. 7 is a detailed environmental side elevation view in section similar to FIG. 6, but showing the diametrically compressed portion of the stent within the stent compression tool of FIGS. 1-2 and over the carrying member.

FIG. 8 is a detailed environmental side elevation view in section, showing the advance of the stent compression tool of FIGS. 1-2 and the stent toward the stent grip and core situated along the carrying member of the stent delivery catheter assembly.

FIG. 9 is a detailed environmental side elevation view in section, showing the stent grip drawing the compressed end of the stent into the stent compression tool of FIGS. 1-2, with the core following and the end of the delivery catheter sheath approaching the tool.

FIG. 10 is a detailed environmental side elevation view in section, showing the end of the delivery catheter sheath fully inserted into the stent compression tool of FIGS. 1-2, with the carrying member and the core and stent grip carried thereon drawing the compressed portion of the stent into the delivery catheter sheath.

FIG. 11 is a detailed environmental side elevation view in section, showing the compressed portion of the stent being drawn further into the delivery catheter sheath by the stent grip as it is drawn into the sheath.

FIG. 12 is a detailed environmental side elevation view in section, showing the carrying member being withdrawn through the lumen of the stent compression tool of FIGS. 1-2, with the stent grip having drawn the compressed stent completely into the sheath.

FIG. 13 is a detailed environmental side elevation view in section, showing the stent compression tool of FIGS. 1-2 being withdrawn over the atraumatic tip of the carrying member, with the fully compressed stent installed within the sheath of the delivery catheter.

FIG. 14 is a detailed environmental side elevation view in section showing the completion of the stent compression process, with the atraumatic tip of the delivery catheter assembly retracted to the end of the sheath.

Similar reference characters denote corresponding features consistently throughout the attached drawings.

### DETAILED DESCRIPTION OF THE PREFERRED EMBODIMENT

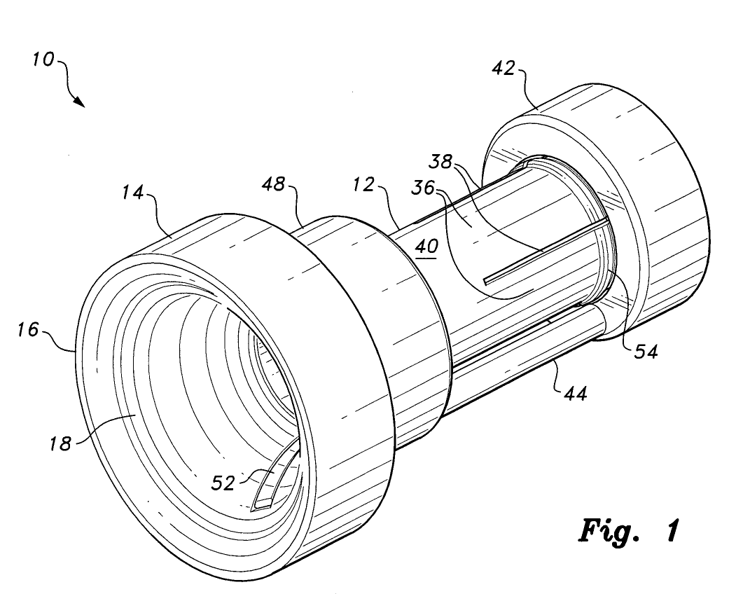

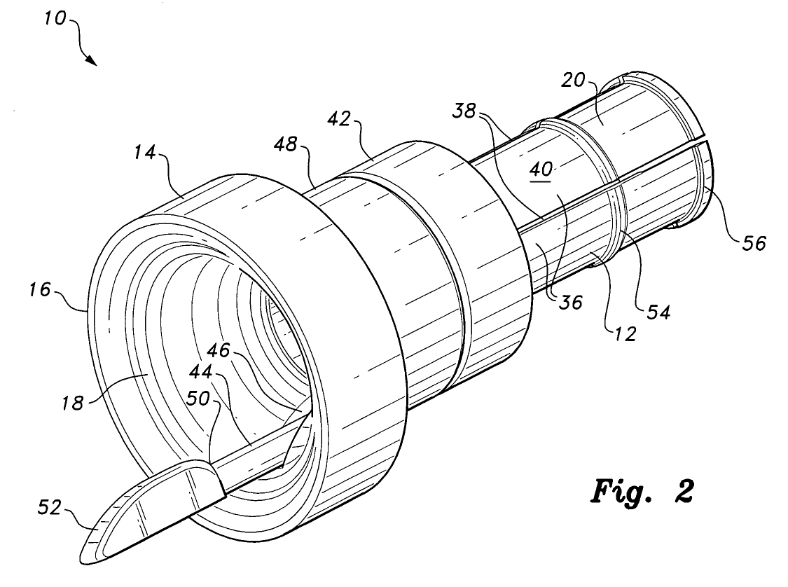

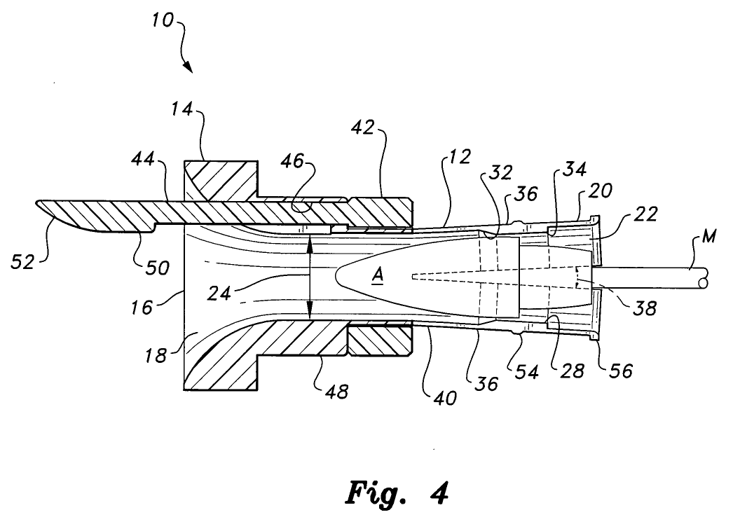

The stent compression tool works in concert with a diametrically compressible stent and conventional stent delivery catheter to compress and install the stent within the delivery catheter. FIGS. 1 and 2 provide perspective views of the stent compression tool 10 with its second portion retaining collar positioned at the second end of the device, and with the retaining collar moved axially toward the opposite first end, respectively. The tool 10 generally comprises a tube 12 having a relatively large diameter first portion 14 and end 16 with a large funnel opening 18 therein, and an opposite smaller diameter second portion 20 with a relatively smaller diameter opening 22 therein, the smaller diameter opening 22 being concentric with the larger diameter funnel opening 18. These features are shown clearly in FIGS. 4 through 13 of the drawings, which illustrate the sequential steps in the compression of the stent and insertion of the stent into the delivery catheter. The funnel opening 18 will be seen to taper smoothly and diametrically to a constant first internal diameter 24, with the second portion 20 of the tube 12 having a constant internal diameter 26 equal to the first internal diameter 24.

A catheter sheath stop 28 (shown in FIGS. 4 through 13) is disposed internally between the first and second internal diameter sections 24 and 26. The sheath stop 28 limits the insertion of the end of the catheter delivery sheath into the tool 10 during the process of stent compression and insertion into the sheath, as discussed further below. The sheath stop 28 comprises a third internal diameter 30 smaller than the first and second internal diameters 24 and 26, with the first end 32 of the stop 28 (i.e., the end toward the funnel opening 18) having a smooth conical taper from the first internal diameter 24 to the smaller internal diameter 30 of the stop. This assures a smooth transition for the stent as it is pushed through the tool 10 from the funnel opening 18, through the sheath stop 28, and toward the smaller diameter end opening 22, as explained further below. The opposite second end 34 of the sheath stop comprises an abrupt diametric planar face or flange 34 having an outer diameter equal to the first and second internal diameters 24 and 26 of the device, and an inner diameter equal to the smaller third diameter 30 of the sheath stop 28. This abrupt planar flange 34 serves as a stop to prevent the insertion of the end of the delivery catheter sheath beyond the stop flange 34, as explained further below.

The tool 10 is formed of a flexible material, e.g., nylon or other durable but flexible plastic. The second portion 20 of the device is divided into a series of longitudinal segments 36 by a corresponding series of longitudinal slots 38 (e.g., four slots forming four segments), with the slots extending along the second portion 20 and into the medial portion 40 of the device. This allows the segments 36 to be spread or expanded radially to allow the passage of an object having an external diameter larger than the internal diameter 30 of the sheath stop 28, for purposes explained further below.

An external circumferential retaining collar 42 is installed about the longitudinal segments 36, and serves to hold the segments closed when the collar 42 is positioned at the opening end of the second portion 20 of the device, as shown e.g. in FIG. 1 of the drawings. The collar 42 is adjustably positionable and may be slid axially toward the first portion 14 of the tool as desired. When the collar 42 is thus adjusted, the segments 36 are free to expand radially to allow an object (e.g., the atraumatic tip of a stent delivery catheter) having a larger diameter than the third internal diameter 30 of the sheath stop 28 to pass therethrough. Once the larger diameter object has cleared the smaller internal diameter 30, the collar may be moved back to its position surrounding the second portion 20 of the tool 10 to hold the segments 36 closed.

The retaining collar 42 includes a collar limit bar 44 extending axially therefrom, outside the tube 12. The limit bar 44 passes through a passage 46 in the larger diameter first portion 14 and an intermediate diameter stop 48 of the tool, and includes a limit stop 50 at the distal end of the limit bar, i.e. the end opposite the collar 42. The limit stop 50 has a larger dimension than the passage 46, and thus precludes separation of the retaining collar 42 from the second end portion 20 and beyond the second end opening 22 of the tool. As the passage 46 extends through the funnel opening 18, the limit stop 50 includes a funnel surface 52 disposed flush with the internal surface of the funnel opening 18 when the collar 42 is positioned at its extreme travel adjacent to the second end opening 22 of the tool.

This allows an object, e.g., a stent, to be inserted into the large diameter funnel opening 18 of the tool without risk of catching or snagging on the limit stop 50. Circumferential first and second collar retaining ridges 54 and 56 are provided around the second portion 20 and the extreme second end opening 22, and have a separation from one another slightly greater than the width of the collar 42. These ridges 54 and 56 serve to hold the collar 42 in position about the second portion 20 of the tool and to hold the second portion segments 36 closed as desired. When it is necessary to pass the tool 10 over a relatively large diameter object, the user merely pushes the collar 42 past the first collar retaining ridge 54 to a position around the medial portion 40 and against the intermediate diameter collar stop 48 of the device, allowing the segments 36 to flex radially outwardly as required.

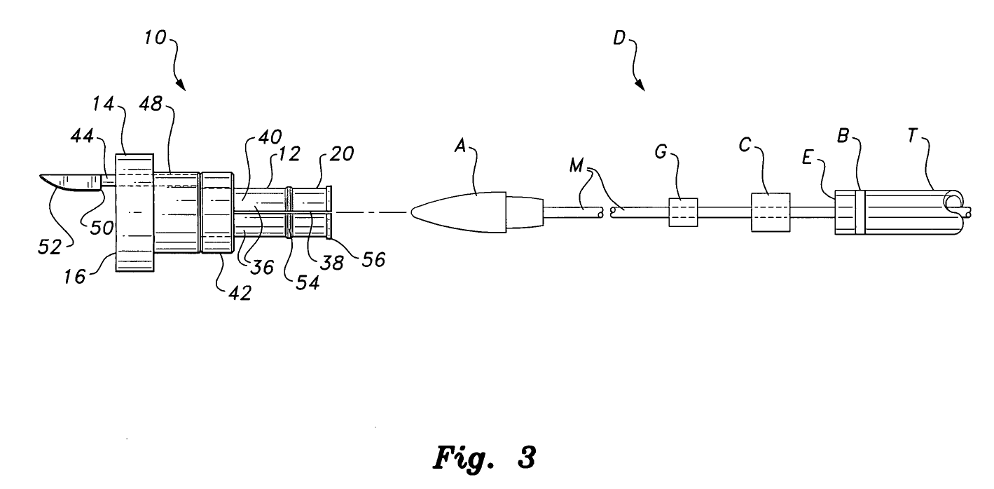

FIGS. 3 through 13 illustrate the progressive steps for compressing a stent and installing the stent within the sheath of a stent delivery catheter by means of the stent compression tool 10. In FIG. 3, a stent delivery catheter D assembly is shown to the right side of the Fig., opposite the stent compression tool 10. The retaining collar 42 of the tool 10 has been moved to its alternative position about the medial portion 40 of the tube 12 and against the intermediate diameter collar stop 48, to allow the longitudinal segments 36 to expand as required.

The delivery catheter D assembly includes a flexible tubular catheter sheath T having a first or distal end E, with a small diameter carrying member M disposed concentrically through the sheath T. The sheath T serves as a stent compression member once the stent has been installed therein. The sheath T includes a radiopaque band B disposed concentrically therearound adjacent the end E thereof, to enable the medical practitioner to locate the end E of the tubular sheath T when installed within a bodily vessel or passage.

A core C is affixed concentrically about the carrying member M, with the core C assisting in locating the carrying member M concentrically within the sheath T. The core C can also assist in holding the stent in place within the bodily vessel or passage as the sheath T is withdrawn over the carrying member M during the installation operation. A stent grip or retention member G is also affixed concentrically upon the carrying member M, but is spaced apart from the core C. The stent grip G is formed of a relatively soft silicone material, or at least coated with such material or a similar material having a surface with a high coefficient of friction. The stent grip G is of slightly smaller diameter than the internal diameter of the sheath T, in order to fit within the compressed stent and press or grip the stent against the inner surface of the tubular sheath T. Finally, an atraumatic tip A is affixed to the distal end of the carrying member M. The atraumatic tip A has an external diameter equal to that of the tubular sheath T, with a relatively rounded blunt tip-to preclude or minimize damage to the bodily vessel or passage as the stent delivery catheter assembly D is inserted and passed therethrough. The above described delivery catheter assembly D with its components T, B, M, C, G, and A are conventional, with the stent delivery tool 10 being configured to fit and work with a given diameter delivery catheter assembly D and stent as required.

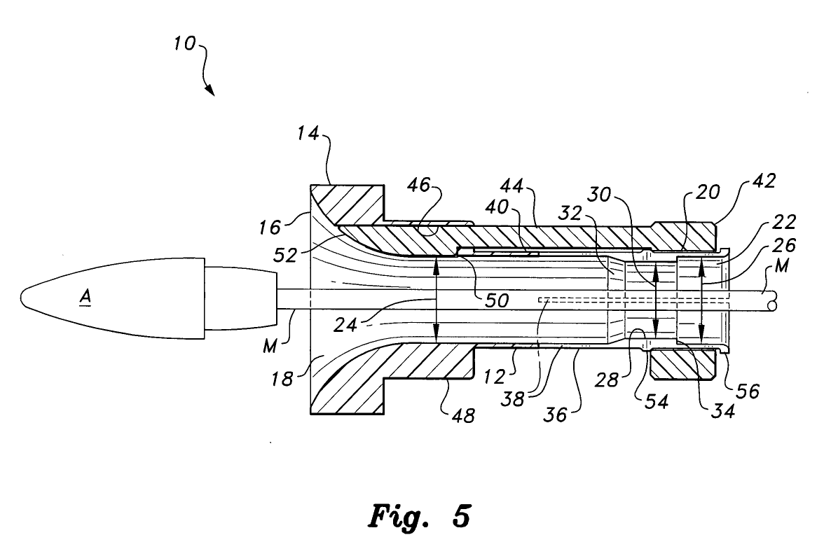

FIG. 4 of the drawings illustrates the first step in the stent compression process, with the relatively large diameter atraumatic tip A being inserted into the smaller diameter opening end 22 of the tool 10. The atraumatic tip A has an external diameter equal to that of the tubular sheath T, with this diameter being about the same as, or perhaps slightly smaller than, the first and second internal diameters 24 and 26 of the tool 10. However, the diameter of the atraumatic tip A (and sheath T) is somewhat larger than the internal diameter 30 of the sheath stop 28 within the tool, when the longitudinal segments 36 are held closed by the collar 42. Hence, it is necessary to move the collar 42 to the position shown in FIGS. 2 through 4 to allow the segments to expand or spread, thus allowing the atraumatic tip A to pass completely through the tool beyond the larger diameter funnel opening 18 to the general position shown in FIG. 5. Once the atraumatic tip A is beyond the sheath stop 28, the collar 42 may be moved back to its position adjacent the second end opening 22 of the tool, as shown in FIG. 5.

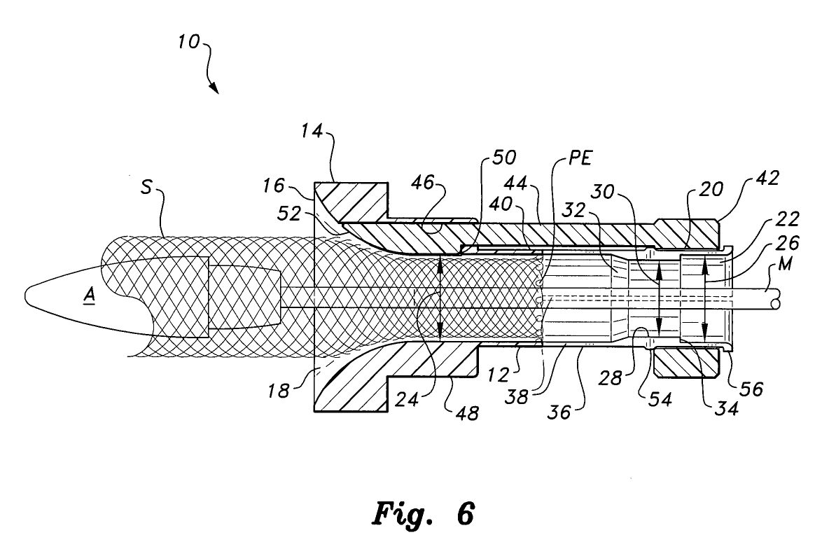

When the atraumatic tip A has been passed through the tool 10 as shown in FIG. 5, the stent S may be passed over the atraumatic tip A and compressed into the tool 10 through the large diameter funnel opening 18 thereof, generally as shown in FIG. 6 of the drawings. The stent S may comprise multiple spirally wound fibers as shown, or other construction as desired. In any case, the stent S has a natural diameter at least equal to that of the vessel or passage into which it is to be installed, with the natural stent diameter also being somewhat greater than the internal diameter of the stent delivery sheath or tube T into which it is being placed. Thus, the stent S must be diametrically compressed to fit into the sheath T, with the stent S later expanding within the bodily vessel or passage in which it is installed, once the sheath T has been removed. In FIG. 6, this stent compression process through the tool 10 is shown partially completed, with the right or proximal end portion PE of the stent S located about halfway through the tool 10 within the relatively smaller first diameter portion 24 and the remainder of the stent S extending from the larger diameter funnel mouth end 18 of the tool.

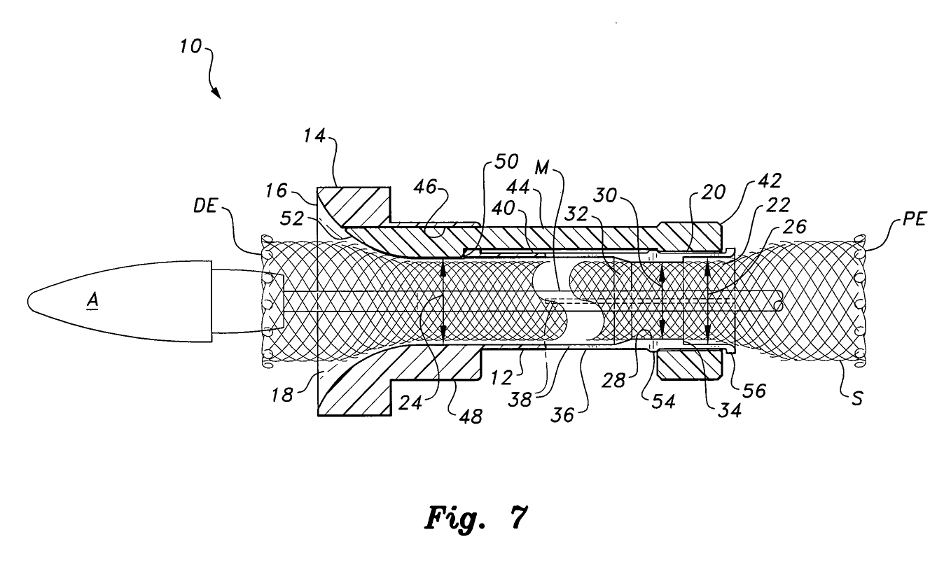

This stent compression process continues as shown in FIG. 7 of the drawings, with the proximal end PE of the stent S having been diametrically compressed through the tool 10 and extending from the small diameter end 22. The plastic material of which the tool 10 is preferably formed provides a low friction surface against which the stent S may slide as it is pushed through and compressed within the tool. It will be seen that the proximal end PE of the stent S has expanded diametrically in FIG. 7 after passing beyond the diametric restriction of the tool 10, as the stent compression process is not yet complete.

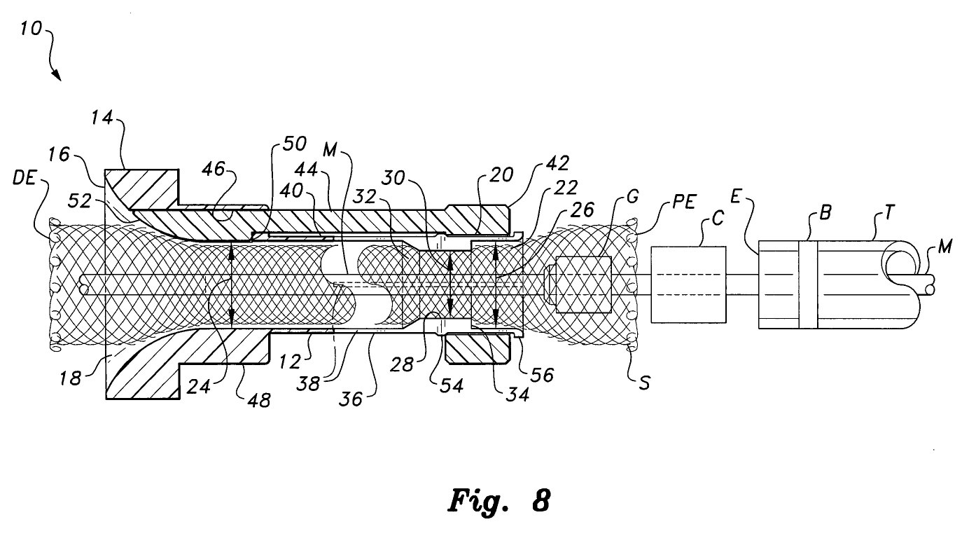

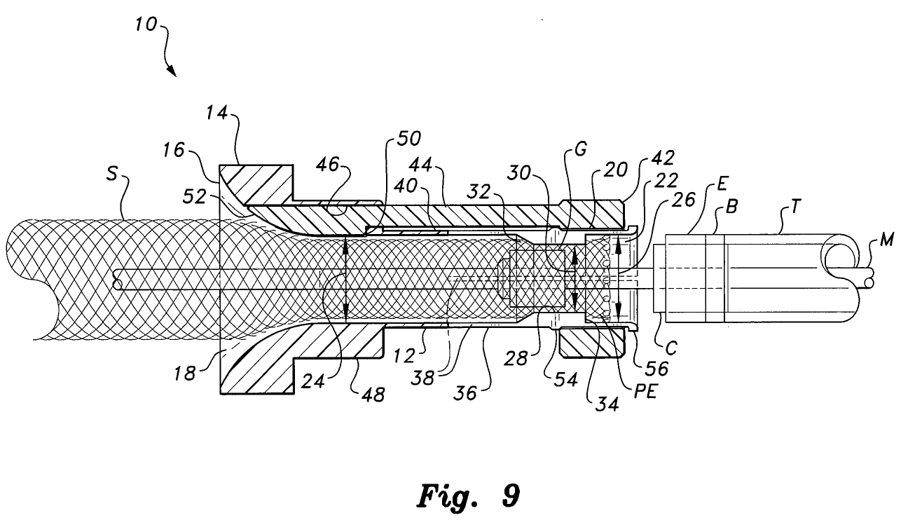

The next step in the stent compression process is shown in FIG. 8, with the tool 10 and the stent S carried therein both moved along the delivery catheter carrying member M toward the stent grip member G and core C affixed to the carrying member. The tool 10 and the stent S are positioned with the proximal end PE of the stent surrounding the stent grip member G. At this point, both the distal end DE of the stent S and the carrying member M are held to preclude movement of the stent relative to the carrying member, and the tool is moved further to the right (as shown in FIGS. 8 and 9) to move the proximal end PE of the stent and the grip member G therein into the smaller diameter second end portion 20 of the tool 10.

The stent grip member G frictionally grips the stent material, with the stent sliding relative to the inner surface of the tool 10. The grip member G has a diameter only very slightly less than that of the narrower third diameter 30 of the sheath stop portion 28 of the tool, and thus grips the stent S firmly between the grip member G and the inner surface of the sheath stop portion 28. The retaining collar 42 remains around the second end portion 20 of the tool, thus preventing the longitudinal segments 36 of the tool from spreading and assuring that the stent S remains tightly secured against the grip member G.

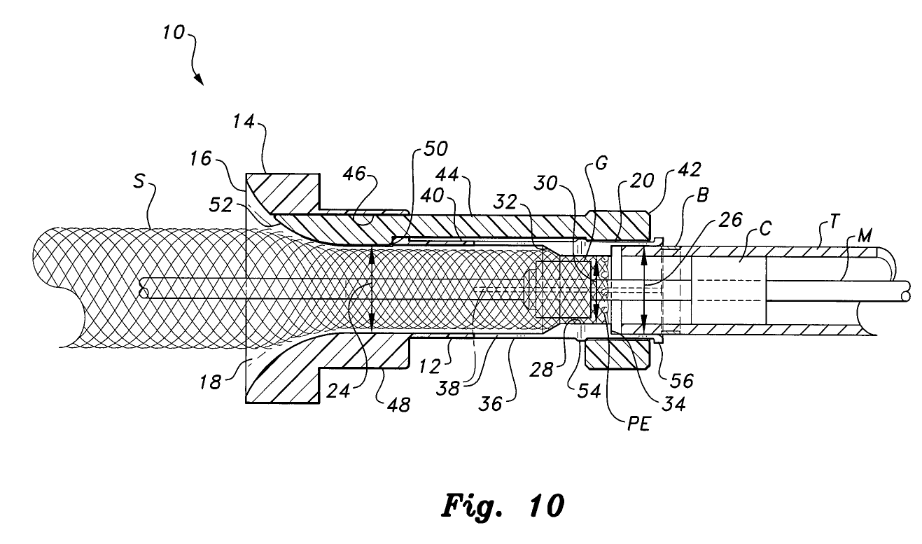

At this point, the proximal end PE of the stent S has been compressed back into the tool 10 just inside the sheath stop portion 28 of the tool. This is shown in FIG. 10 of the drawings. As the second end portion 20 of the tool is now open, excepting the carrying member M, the end E of the stent delivery sheath tube T may be installed within the second end 20 of the tool. In FIG. 10 the insertion of the stent delivery tube T is nearly complete, but has not quite reached the diametric flange or ring 34 forming the abrupt end of the sheath stop portion 28.

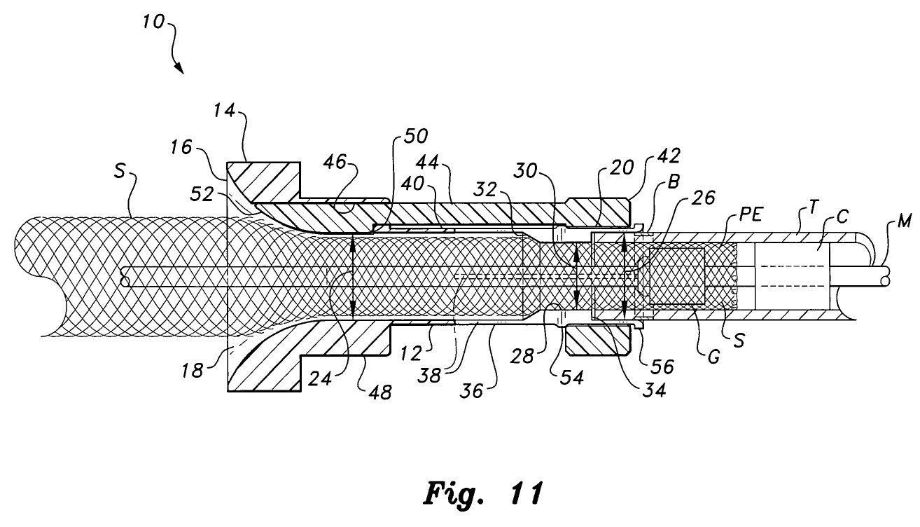

FIG. 11 illustrates the next step in the process, with the end E of the stent delivery sheath T having been pushed into the second end portion 20 of the tool 10 essentially as far as possible, with further insertion being stopped by the sheath stop flange 34 forming the second end of the sheath stop 28. The outer diameter of the sheath tube T is essentially equal to the inner diameter 26 of the second end portion of the tool 10, while the inner diameter of the sheath T is essentially equal to the inner diameter 30 of the sheath stop 28. This allows the stent S to continue to move into the delivery catheter sheath tube T as the carrying member M and its stent grip member G are drawn back to the right (in the orientation shown in FIG. 11).

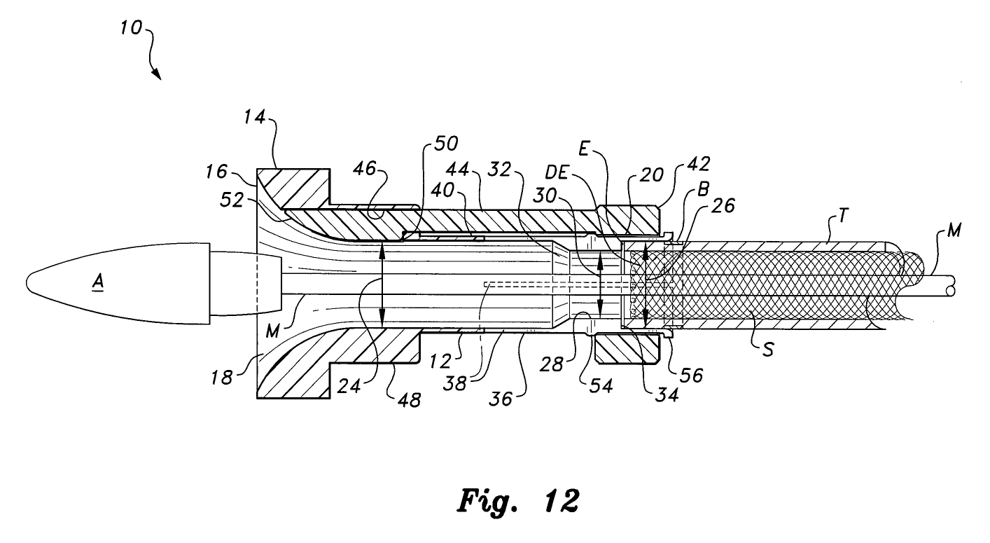

The process of inserting the stent S completely into the stent delivery sheath tube T has been completed in FIG. 12, with the carrying member M having been drawn further to the right to cause the grip member G to draw the stent S farther into the delivery sheath or tube T. The atraumatic end A is adjacent to, or slightly within, the larger diameter funnel end 18 of the tool 10 at this point, with the stent S being completely compressed and positioned entirely within the delivery sheath tube T.

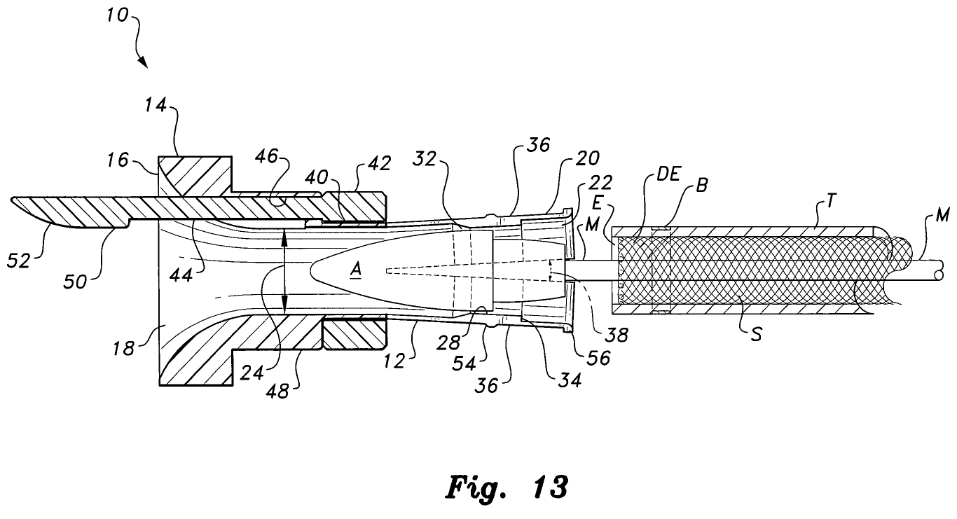

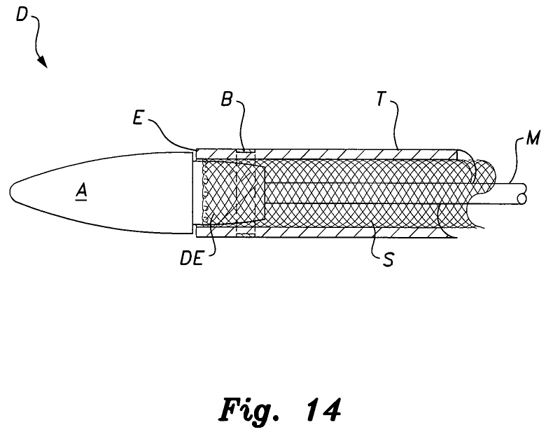

FIG. 13 illustrates the penultimate step in the compression and installation of the stent within its delivery catheter sheath. In FIG. 13, the retaining collar 42 has been repositioned over the medial portion 40 of the tool, adjacent to the intermediate retaining collar stop 48. This allows the longitudinal segments 36 to spread or expand as the atraumatic tip A is pulled back through the tool 10 from the larger diameter first end 16 to the smaller diameter second end opening 22. Once the atraumatic tip A has been removed from the tool 10 (or the tool removed from the atraumatic tip), the retaining collar 42 is returned to its position surrounding the second end portion 20 of the tool to retain the longitudinal segments, as shown in FIG. 1. The carrying member M is drawn further to the right to position the atraumatic tip A at the end E of the stent sheath tube T, as shown in FIG. 14, to complete the process of stent compression and stent installation into the delivery sheath.

Accordingly, the stent compression tool greatly facilitates the compression and installation of a compressible stent into a delivery sheath prior to use. This procedure has heretofore been a tedious task that requires a fair amount of time and expertise to accomplish properly. The stent compression tool removes much of the exacting labor previously required for this task, reducing the task essentially to drawing and removing various sheath components and the stent into and from the tool. The end result is a properly compressed stent free of kinks or damage, positioned within the delivery catheter, ready for placement within the patient.

It is to be understood that the present invention is not limited to the embodiment described above, but encompasses any and all embodiments within the scope of the following claims.

We claim:

1. A stent compression tool, comprising a tube defining a lumen, the tube having:
a first portion defining a large funnel opening tapering diametrically to a constant first internal diameter;
a second portion opposite the first portion, the second portion defining a small opening therein concentric with the first opening, the second portion having a constant second internal diameter equal to the first internal diameter; and
a catheter sheath stop extending into the lumen between the first internal diameter and the second internal diameter.

2. The stent compression tool according to claim 1, wherein the catheter sheath stop defines:
a third internal diameter smaller than the first and second internal diameters;
a first end having a smooth conical taper from the first internal diameter; and
a second end having an abrupt diametric planar face transitioning from the third internal diameter of the sheath stop to the second internal diameter of the second portion.

3. The stent compression tool according to claim 1, wherein the tube is formed of flexible material and has a medial portion disposed between the first portion and second portion, the second portion having longitudinally disposed slots extending from the second portion into the medial portion to define a plurality of longitudinal segments, the tool further comprising an adjustably positioned retaining collar disposed externally about the longitudinal segments.

4. The stent compression tool according to claim 3, further comprising:
a collar limit bar extending from the retaining collar external to the tube, the first portion having a passage defined therein, the collar limit bar extending through the passage; and
a limit stop disposed upon the limit bar opposite the retaining collar, the limit stop precluding axial travel of the retaining collar beyond the second portion opening.

5. The stent compression tool according to claim 4, wherein the limit stop has a funnel-shaped surface flush with the funnel opening when the retaining collar is positioned at the second end of the tube and the second portion of the tube has circumferential first and second collar retaining ridges disposed thereabout, the ridges precluding inadvertent travel of the retaining collar from the second portion.

6. A method of compressing a stent into a delivery catheter using the apparatus of claim 1, comprising the steps of:
(a) inserting an atraumatic tip of a delivery catheter through the tool from the small opening to the large opening of the tube;
(b) passing the stent over the atraumatic tip of the delivery catheter and into the large opening of the tool;
(c) compressing the stent diametrically through the second portion of the tool;
(d) inserting a grip member of the delivery catheter assembly into the stent and pushing the stent into the second portion of the tool using the grip member;
(e) inserting a sheath of the delivery catheter into the second portion of the tool;
(f) drawing the stent into the sheath of the delivery catheter using the grip member;
(g) withdrawing the tool from the delivery catheter and past the atraumatic tip of the catheter; and (h) positioning the atraumatic tip at the end of the sheath of the delivery catheter.

7. A stent compression tool, comprising:
a tube formed of flexible material, the tube defining a lumen and having:
a first portion defining a large diameter cylinder and a large funnel opening tapering diametrically to a constant first internal diameter;
a medial portion; and
a second portion opposite the first portion, the second portion defining a small opening and a constant second internal diameter equal to the first internal diameter, the medial portion being disposed between the first and second portions, the second portion having a plurality of slots defining a plurality of longitudinal segments into the medial portion; and
an adjustably positioned retaining collar disposed externally about the second portion.

8. The stent compression tool according to claim 7, further comprising:
a collar limit bar extending from the retaining collar external to the tube, the first portion having a passage defined therein, the collar limit bar extending through the passage; and
a limit stop disposed upon the limit bar opposite the retaining collar, the limit stop precluding axial travel of the retaining collar beyond the second portion opening.

9. The stent compression tool according to claim 8, wherein the limit stop has a funnel-shaped surface flush with the funnel opening when the retaining collar is positioned at the second end of the tube and the second portion of the tube has circumferential first and second collar retaining ridges disposed thereabout, the ridges precluding inadvertent travel of the retaining collar from the second portion.

10. The stent compression tool according to claim 7, wherein the lumen has a narrow diameter portion smaller than the first and second diameters, the narrow diameter portion forming a catheter sheath stop.

11. The stent compression tool according to claim 10, wherein the cable sheath stop has a first end having a smooth conical taper from the first internal diameter to the narrow diameter portion, and a second end forming an abrupt diametric planar face transitioning from the narrow diameter portion to the second internal diameter of the second portion.

12. A method of compressing a stent into a delivery catheter using the apparatus of claim 7, comprising the steps of:
(a) inserting an atraumatic tip of a delivery catheter through the tube from the small opening to the large opening thereof;
(b) passing the stent over the atraumatic tip of the delivery catheter and into the large opening of the tube;
(c) compressing the stent diametrically through the small diameter portion of the tube;
(d) inserting a grip member of the delivery catheter into the stent and pushing the stent into the tube using the grip member;
(e) inserting a sheath of the delivery catheter into the small diameter end of the tube;
(f) drawing the stent into the sheath of the delivery catheter using the grip member;
(g) withdrawing the tool from the delivery catheter and past the atraumatic tip thereof; and
(h) positioning the atraumatic tip at the end of the sheath of the delivery catheter.

13. A method of compressing a stent using a stent compression tool applied to a stent delivery catheter assembly, comprising the steps of:
(a) inserting an atraumatic tip of the delivery catheter through the tool from a small opening to a large opening of the tool;
(b) passing the stent over the atraumatic tip of the delivery catheter and into the large opening of the tool;
(c) compressing the stent diametrically through a small diameter portion and end of the tool;
(d) inserting a grip member of the delivery catheter assembly into the stent and pushing the stent into the tool using the grip member;
(e) inserting the sheath of the delivery catheter into the small diameter end of the tool;
(f) drawing the stent into the sheath of the delivery catheter using the grip member;
(g) withdrawing the tool from the delivery catheter and past the atraumatic tip thereof; and
(h) positioning the atraumatic tip at the end of the sheath of the delivery catheter.
